(19) United States
(12) Patent Application Publication
(10) Pub. No.: US 2006/0051760 A1
(43) Pub. Date: Mar. 9, 2006

(54) Compositions and methods used for identifying factors required for the agglomeration of proteins

(76) Inventor: Abraham Grossman, Pleasantonville, NY (US)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/524,681

(22) PCT Filed: Aug. 13, 2003
(86) PCT No.: PCT/US03/25470

§ 371 Date: Feb. 15, 2005

Related U.S. Application Data
(60) Provisional application No. 60/403,726, filed on Aug. 15, 2002.

(51) Int. Cl. C40B 40/08; C12Q 1/68

(52) U.S. Cl. 435/6

(57) ABSTRACT

Described herein are compositions and methods for identifying cellular factors involved in protein agglomeration. One such factor is a nucleic acid component. Another factor is a cellular macromolecular co-binding component. The presence of certain NAs and co-binding factors facilitate the agglomeration of certain proteins. The compositions and methods described herein are employed to identify this factors involved in the protein agglomeration process. Moreover, methods for assessing the efficacy of a pharmaceutical agent in protecting prion proteins is also described herein.

### FIELD OF THE INVENTION

The present invention pertains to compositions and methods used to interact with proteins. In particular, this invention is directed toward compositions and methods used to identify cellular factors involved in the misfolding of proteins.

### BACKGROUND OF THE INVENTION

Essentially there are two types of nucleic acid found in living cells. One is deoxyribonucleic acid (“DNA”), and the other is ribonucleic acid (“RNA”). Under normal physiological conditions, both of these nucleic acid molecules are associated with proteins and form nucleoprotein complexes. These proteins can include scaffolding proteins, enzymes, ligases, telomerases, etc. These nucleic acid binding proteins perform functions necessary for normal metabolism and cell/tissue viability.

A significant portion of RNA-binding proteins (“RNP”) mediate post-transcriptional regulation of gene expression. Heterogenous nuclear RNAs (“hnRNA”) are the primary transcripts of protein encoding genes. These transcripts (hnRNA) are processed in the nuclei of eukaryotic cells and, at least a portion of such hnRNAs, become messenger RNAs (“mRNAs”). From the time hnRNAs emerge from the transcriptional complex, and throughout the time they are in the nucleus, they are associated with proteins termed hnRNA proteins. Members of this family of proteins are required for multiple steps during mRNA metabolism, including pre-mRNA processing and MRNA localization, translation and stability. The majority of proteins associated with RNAs appear to be associated with hnRNAs and mRNAs in hnRNP and mRNA complexes.

Some disease processes appear to stem from the misfolding of proteins. This misfolding is often associated with nucleic acids (NAs) and cellular factors. These misfolded proteins can go on to form pathological agglomerations. These agglomerations have been shown to be associated with neuronal cell death and brain wasting diseases such as Alzheimer's and Parkinson's disease in humans, scrapie, mad cow disease and chronic wasting diseases in animals. Spongiform encephalopathies, often involved with certain neuronal cell death and brain wasting syndromes, characteristically have protein plaques or agglomerations made manifest upon dissection. In spongiform encephalopathies, prion proteins are thought to be the etiologic agent. Prion-based diseases result from “infectious proteins” that are cellular benign prion proteins misfolded into an infectious isoform. This infectious isoform is involved in pathological protein agglomeration. Misfolded proteins also appear to damage cells in the lung, heart, kidney, pancreas and other organs.

It is believed that these misfolded proteins that result in agglomeration-causing disease are associated with certain RNA molecules and other cellular factors. Currently, a need exists for compositions and methods that can identify these cellular factors involved in protein misfolding.

### SUMMARY OF THE INVENTION

The present invention pertains to compositions and methods for identifying cellular factors that physiologically facilitate protein agglomeration. One such factor is a nucleic acid (hereinafter “NA”) component comprising different species of DNA and RNA. Another factor is a macromolecular cellular binding factor hereinafter CBF). The presence of certain NAs and CBFs facilitate the agglomeration of proteins, this agglomeration process, as mentioned above, has been associated with pathological physiology. This invention provides compositions and methods for identifying such CBFs involved in this process. Moreover, the present invention is directed to methods for examining the efficacy of pharmaceutical agents in their ability to affect protein agglomeration. Further, this invention pertains to a kit that can be employed for testing pharmaceutical agent that putatively affect protein agglomeration.

In one embodiment, a method for identifying the presence or absence of one or more CBFs in a sample matrix is disclosed. This method comprises taking an aliquot from the sample matrix and to this sample matrix adding a predetermined NA and a predetermined native protein. The admixture is incubated under conditions suitable to allow for the agglomeration of the added protein. Preferably, the agglomeration complex comprises a protein component (“A”), a CBF component (“B”), and a NA component (“C”) in which the complex can be represented by the formula [AₓB_{y}C_{z}], wherein x, y, and z are integers, each integer independently having a value from one (1) to infinity (O). The protein agglomerations can then be detected, for example, using the insolubility property of the nucleo-protein complex, or by electron microscopy, or by other methods well known to the skilled practitioner. A positive result obtained from these detection methods is indicative indicates of the presence of one or more CBFs in the original sample matrix.

An additional detection method comprises augmenting the method articulated above with the presence of protease K and examining protein resistance to digestion. In the absence of a CBF, certain proteins like prion proteins are free and soluble, as a consequence, the proteins are readily digested by proteinase K. Following the incubation of sample matrix, NA and protein, protease K is added to the admixture. After allowing for a suitable incubation period, the reaction product is subjected to protease K treatment. The presence of prion protein fragment following protease K treatment is examined by, for example, gel electrophoresis together with Western blot analysis. If the prion protein is bound in an agglomeration complex, then it is not susceptible to digestion by protease K, hence upon examination there should be a negative finding of prion protein fragments thereby indicating the presence of one or more CBFs in the original sample matrix.

In another embodiment of the present invention, a method for examining the efficacy of a pharmaceutical agent with respect to its ability to inhibit protein agglomeration is disclosed. Sensitivity to protein digestion by a prion protein can be used to illustrate its proclivity towards participating in protein agglomeration. Insensitivity toward protein digestion using, for example, protease K, suggests that a prion protein's conformation has change in such a manner so as to facilitate protein agglomeration. The prion protein in the agglomeration complex is refractory to protein digestion. Conversely, if the prion protein is susceptible to protein digestion, then it must be free and soluble and therefor, not participating in an agglomeration complex. This understanding provides the basis of an assay system that can be used to assess the efficacy of a putative pharmaceutical agent in inhibiting protein agglomeration. Simply, if in the presence of a test agent (as well as NA and CBF) the prion protein is found to be susceptible to protein digestion, then this suggests that the test agent inhibited the required conformational change by the prion protein necessary for protein agglomeration.

This method comprises forming an admixture of one or more CBFs, one or more NA antibodies, one or more prion proteins, and a test pharmaceutical agent. A protease enzyme is added to this admixture, such as protease K, under conditions suitable for protein digestion. After a suitable period of time, an aliquot is retrieved from the admixture reaction vessel and examined viz. the status of the native prion protein. If upon examination digestion products of the native prion protein are detected, then this is a positive result indicating that the pharmaceutical test agent is effective in protecting the prion protein from forming an agglomeration complex. If on the other hand, an agglomeration complex forms, then the drug is obviously ineffective in preventing such a complex formation at the level of the prion protein.

The present invention pertains to an assay kit. This kit can be used to assess a test agent's ability to prevent protein agglomeration. The kit comprises one or more NA antibodies, one or more CBFs, and one or more prion proteins. The kit can additionally comprise protease K. Preferably, this kit is used in conjunction with the above-described method.

### BRIEF DESCRIPTION OF THE DRAWINGS

FIG. 1 is the amino acid sequence for fibronectin;

FIG. 2 is a gel demonstrating the resistance of prion protein to proteinase K in the presence of a CBF and NA antibody;

FIG. 3 depicts two gels demonstrating the presence of a CBF in human serum; and

FIG. 4 depicts a gel electrophoresis illustrating the protection of a prion protein from protein digestion in the presence of chlorpromazine.

### DETAILED DESCRIPTION

The present invention pertains to compositions and methods for identifying cellular factors involved in protein agglomeration. One such factor is a nucleic acid (NA) component. Another factor is a cellular binding factor (CBF) component. The presence of certain NAs and CBFs in a mixture containing proteins like prion proteins will facilitate the agglomeration of these proteins. Previous work has elucidated the NA component capable of participating in this process. This invention provides compositions and methods useful for identifying CBFs involved in this agglomeration process. Further, this invention also pertains to methods for assessing the efficacy of a putative pharmaceutical in preventing protein agglomeration formation. Moreover, a kit is described herein that can be used by practitioners for examining test agents and their ability to prevent protein agglomeration formation.

The invention described herein pertains in part to functional ligands and their ability to interact with target molecules. A functional ligand, as used herein, is a molecule that when bound to or interacting with a target molecule, chemically and/or biologically modifies the target molecule. For example, the interaction of a functional ligand with a target molecule can confer protein digestion resistance upon the bound target molecule in contrast to its free form that is otherwise susceptible to protease digestion. Other chemical and biological properties can be modified as well by such interaction. Many of these modifications can be detected by methods well known to those skilled in the art employing techniques extant in the art. Examples of such functional ligands include, but are not limited to, nucleic acids, peptides, proteins, lipoproteins, glycoproteins, and combinations thereof.

Protein monoclonal, polyclonal antibodies and nucleic acid molecules are currently the functional ligands of choice for detecting the presence of target molecules in a sample matrix.

Antibodies developed in laboratory animals are immunogenic, meaning that they will cause an immune response in humans. Therefore, antibodies cannot be used as therapeutics without “humanization,” an additional, highly sophisticated and very expensive modification process. The potential to induce undesirable immune reactions dramatically reduces the therapeutic application of antibodies. Therefore, only a few out many hundreds of antibodies developed so far have been accepted for therapeutic purposes. Thus, antibody technology has the disadvantages of being labor intensive, costly in time and expense, and needs to overcome immunogenic responses for therapeutic applications. Additional undesirable limitations of antibodies are their poor sensitivity (the ability to identify a small number of molecules in a sample) and variable specificity (the ability to differentiate between benign and pathological molecules). Despite the many limitations, new antibodies are regularly being developed for new target molecules.

Nucleic acid probes are similar in many respects to antibodies, however their application is quite different. Nucleic acid probes are designed to identify, bind and inactivate NA target molecules. Nucleic acid probes lack many limitations observed with monoclonal antibodies. The major limitation of NA probes is that they are designed specifically for the detection of DNA and RNA targets and require special modification to detect protein and other non-NA cellular targets. This limitation of NAs is very important, because most proteins are thousands of times more abundant than DNA or RNA products. Very often protein targets are easier to access than NA molecules, for example, protein detection does not require laborious sample preparation. The relative high stability of proteins during sample preparation and their potential higher physiological relevance compared with DNA or RNA made them targets of choice in the development of detection assays. Some NAs, such as ribosomal or mitochondrial RNAs, are present in multiple copies. However, viruses, one of the most important classes of pathogens, do not have ribosomal or mitochondrial RNAs. Additionally, not all diseases require a pathogenic organism or its NA, for example, toxins, metabolic abnormalities, or poisonous gas. Use of NA as gene-therapy procedures for the inactivation of NA targets is still very problematic, moreover, there is not one presently posited gene-therapy method approved by FDA.

The current invention employs NAs as functional ligands. Specifically, these NAs can be characterized as antibody-like (hereinafter “NA antibody”) for they specifically interact with target molecules, usually proteins. (NA antibodies described herein are available from Q-RNA, New York, N.Y.) NA antibodies can be either DNA or RNA molecules that comprises a nucleotide sequence required to interact with a particular target molecule. The target molecule can include a protein or protein fragment. These NA antibodies can be directed at any number of targets. For example, they can be directed toward prion proteins involved in neurological diseases, such as spongiform enchephalitis. (See PCT/US02/16922, filed May 30, 2002, the entire teachings of which is incorporated herein by reference.) Nucleic acid antibodies can comprise artificial, synthetic nucleotide sequences that a skilled practitioner can insert by methods well known in the art thereby forming chimeric NA molecules that are designed specifically for a particular target.

The degree of molecular discrimination achieved by a NA antibody in recognition of the corresponding target matches and, in some instances, surpasses that of traditional monoclonal antibodies. An important advantage of a NA antibody over their protein counterpart, e.g., monoclonal antibodies, is that a NA antibody's composition and structure can be very easily modified to enhance its performance. At present, it is widely accepted that NA antibodies represent new and powerful molecular biological tools with potentially wide applications in medicinal diagnosis, biotechnology and therapeutics.

The roles of NA antibodies in the discovery of new drugs, the design of diagnostic devices and therapeutics, as well as in basic and applied biotechnology research, is enormous. The unique properties of NA antibodies, with their specific and tight binding activities, create new opportunities to construct extremely powerful molecular biological tools that can be applied to various practical purposes, as well as further our understanding of biological processes.

In one aspect of the present invention, the NA antibody is an RNA molecule. RNAs are unique molecules in the universe of macromolecules; they combine features possessed by both DNA and proteins. RNA, like DNA, are NA, but like proteins, RNA can fold into a variety of stable secondary structures that in a “lock and key” fashion can interact and form complexes with complimentary structures from other molecules. It has been shown, without any known exception, that the specific interaction of RNAs with non-NA targets is determined by their nucleotide composition and by the shape of the binding regions of both the RNA and target molecules. High affinity RNAs (with Kd's ranging from 0.1-1.0 nM) have been successfully identified for a large number of targets, ranging in chemical composition and size from small organic molecules to highly complex multimeric structures, such as viruses.

Nucleic acids bind to protein molecules. This binding is often facilitated by the secondary structure assumed by the NA. For example, loops and bulges of nucleotides are often involved in protein binding. Protein agglomeration is often facilitated by such NA interaction. Proteins that interact with RNA (hereinafter referred to as “RNP”) typically have RNA-binding motifs that will receive the proper RNA secondary structure. Under suitable conditions, RNPs can non-specifically bind to RNA having gross features that are recognized by the protein without regard to particular nucleotides or nucleotide sequences. It is generally believed that a nucleic acid's higher ordered structure is what provides binding recognition to the protein. For example, RNA is notorious for possessing secondary structures like loops that may in turn serve as structural motifs used for binding with RNPs. (This notion is amenable to analysis simply by taking a primary nucleotide sequence of an RNA molecule that binds to a particular protein and changing the nucleotide sequence of a particular secondary structure, like a loop, in order to knock-out the structure and determine the binding avidity between the mutated RNA and protein.)

Stringent in vitro assays have demonstrated that heterogeneous nuclear RNA-binding proteins (hnRNP) have different preferences for specific RNA sequences. One study examined the binding of various hnRNAs to various RNAs under 2M NaCl conditions resulting in a finding of striking avidity of hnRNPs for their preferred RNAs. These studies indicate that different hnRNP, and apparently other RNPs, discriminate among and between different RNAs. This property of discrimination can be exploited in the isolation, purification and classification of various groups of RNPs.

The molecular architecture of RNPs was studied in detailed using hnRNPs. However, the general principles gained through studying the hnRNP systems are applicable to other RNPs. Most RNPs have a modular structure with one or more RNA-binding domains (RBD) and special domains that mediate interaction with another protein. The hallmarks of RBDs in RNPs are distinct consensus sequences separated from each other by stretches of approximately thirty amino acids. Most of the amino acids that are involved in binding RNA are located in ∃-pleated sheets. These particular structural elements of RBDs appear to provide an exposed surface that can serve as a platform to which an RNA molecule can bind. The RNA, when bound, remains exposed (as opposed to being buried within a pocket of the protein) and thus accessible to other proteins. Many RNPs contain more than one RBD and can therefore bind to multiple RNA sequences or interact with multiple RNA molecules simultaneously.

Nucleic acid antibodies of the present invention comprise one or more RNA or DNA molecules having affinity for at least one protein involved in protein agglomeration. The NA component is a naturally or non-naturally occurring molecule with twenty or more nucleotide bases. In one embodiment, at least one nucleotide sequence portion of this RNA molecule has affinity to at least one consensus sequence present in the agglomeration RNA-binding protein. A “consensus sequence” of the present invention refers to an RNA-binding motif present in a protein that recognizes single-stranded RNA secondary structural elements such as hairpin loops, bulge loops, internal loops, or single-stranded regions. In one embodiment of the present invention, the portion of RNA polynucleotide having affinity for agglomeration proteins is a sequence that is derived from either an RNA virus, an RNA phage, a messenger RNA (“mRNA”), a ribosomal RNA (“rRNA”), a transfer RNA (“tRNA”), a sequence that is received as a template by one or more RNA dependent RNA polymerases, or a combination thereof.

The terms “virus” and “phage” will be used interchangeably throughout this disclosure to mean “virus and phage”. In one aspect of the invention, the RNA virus is a retrovirus. The RNA virus can be selected from the group consisting of a human immunodeficiency virus (HIV), polio virus, influenza virus, smallpox virus, chicken pox virus, Herpes virus, varicella zoster virus, Epstein-Barr virus, cytomegalovirus, feline leukemia virus (FeLV), human T cell leukemia virus (HTLV), simian immunodeficiency virus (SIV), and combinations thereof.

The RNA template of the present invention is an RNA that can be received and amplified by Q-beta replicase, Q-Amp, and nucleic acid replicases for DNA or RNA. (See PCT/US02/16922.) The skilled artisan will appreciate that any RNA dependent RNA polymerase (“RNA pol”) that will amplify the NA antibodies of the present invention are within the scope of this invention. In one embodiment of the instant invention, the RNA template is RQ11+12, the sequence of which is (SEQ ID NO 1):

5′GGGGUUUCCAACCGGAAUUUGAGGGAUGCCUAGGCAUCCCCCGUGCGU
CCCUUUCGAGGGAUUGUCGACUCUAGUCGACGUCUGGGCGAAAAAUGUAC
GAGAGGACCUUUUCGGUACAGACGGUACCUGAGGGAUGCCUAGGCAUCCC
CCGCGCCGGUUUCGGACCUCCAGUGCGUGUUACCGCACUGUCGACCC 3′

Other RNA molecules of the present invention include, but are not limited to, midi-variant RNA (MDV RNA), mini-variant RNA (MNV RNA), MNV-AP1 RNA, MNVUP RNA, MNVLO RNA, RQ RNA, and combinations thereof. Their respective sequences are:

The DNA sequence encoding MDV RNA (SEQ ID NO 2) is:

5′GGGGACCCCCCGGAAGGGGGGACGAGGTGCGGGCACCTCGTACGGGAG
TTCGACCGTGACGAGTCACGGGCTAGCGCTTTCGCGCTCTCCCAGGTGAC
GCCTCGTGAAGAGGCGCGACCTTCGTGCGTTTCGGCGACGCACGAGAACC
GCCACGCTGCTTCGCAGCGTGGCCCCTTCGCGCAGCCCGCTGCGCGAGGT
GACCCCCGAAGGGGGGTTCCCCA 3′

The DNA sequence encoding MNV RNA (SEQ ID NO 3) is:

5′GGGTTCATAGCCTATTCGGCTTTTAAGGACCTTTTTCCCTCGCGTAGC
TAGCTACGCGAGGTGAC CCCCCGAAGGGGGGTGCCCC 3′

The DNA sequence encoding MNV-AP1 RNA (SEQ ID NO 4) is:

5′GGGTTCATAGCCTATTCGGCTTCGCGCATGGGAATTTGAGGGACGATG
GGGAAGTGGGAGCGCGTTTTAAAGGACCTTTTTCCCTCGCGTAGCTAGCT
ACGCGAGGTGACCCCCCGAAGGGGGGTGCCCC 3′

The DNA sequence encoding MNVUP RNA (SEQ ID NO 5) is:

5′GGGTTCATAGCCTATTCGGCTTCGCGCCCGTTTATAATACTTAGTGAG
CGCGTTTTAAAGGACCTTTTTCCCTCGCGTAGCTAGCTACGCGAGGTGAC
CCCCCGAAGGGGGGTGCCCC 3′

The DNA sequence encoding MNVLO RNA (SEQ ID NO 6) is:

5′GGGTTCATAGCCTATTCGGCTTCGCGCCCCTGGGGTTTGCCTCAGGAG
CGCGTTTTAAAGGACCTTTTTCCCTTGCGTAGCTAGCTACGCGAGGTGAC
CCCCCGAAGGGGGGTGCCCC 3′

The RNA sequence can be obtained using DNA that encodes for the RNA sequence. The DNA can be inserted within a suitable vector followed by transfecting a suitable host with the vector using methods well known to those skilled in the art. The transcripts can then be isolated by methods commonly known in the art.

Nucleic acid antibodies can be seen as universal detector molecules. Neither monoclonal antibodies nor nucleic acid probes are universal in their application. Antibodies usually target proteins, whereas nucleic acid probes are used for detection of nucleic acid targets. Nucleic acids are easily modified for signal detection, whereas monoclonal antibodies require an additional enzyme-based component to produce a signal. The universal nature of NA antibodies allow for detection of both non-nucleic acid molecules as well as for hybridization with nucleic acid targets. The universe of non-NA targets includes proteins, toxins, and small bioregulators against which antibodies are practically impossible to develop.

In contrast to traditional monoclonal antibodies, NA antibodies can be easily modified to increase their avidity to targets and enhance their specificity. Nucleic acid antibodies can be used for detection of designated targets in formats that employ existing immunodiagnostic or nucleic acid assay hardware. Importantly, they can be used together with traditional antibodies to dramatically increase the total affinity of extant immunodiagnostic assays.

Functional ligands like NA antibodies have potential as a highly specific molecular therapeutic. It has been demonstrated that the interaction of a constructed prion-specific NA antibody with a soluble, proteinase K digestible form of the prion protein makes this protein non-soluble and proteinase K resistant. This interaction of functional ligands can be exploited for identifying additional metabolite(s) and cell component(s) that might represent additional groups of functional ligands with a special interest for future therapeutic purposes related to prion diseases. Specifically, the present inventors demonstrated that this functional interaction is dependent upon at least one other functional ligand, namely a cellular binding factor (CBF). (The present invention encompasses one or more CBFs, however, for convenience and simplicity the singular form of CBF is used throughout unless stated otherwise). It is apparent that the functional ligand CBF, identified with the help of another functional ligand, specifically a prion protein specific NA antibody, is a potential target for therapeutic application. Nucleic acid antibodies can play an important role, not only in identifying new targets for therapeutics, but also as therapeutics themselves. Inactivation of CBF using a modified NA antibody would prevent prion proteins from agglomerating, hence blocking an important part in the overall prion-based disease process.

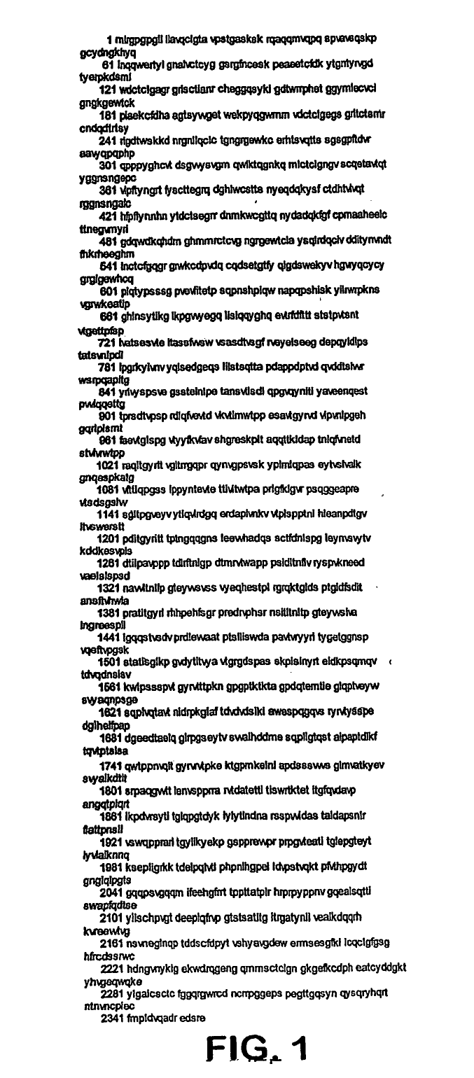

It appears that this previously unknown cellular component, CBF, participates in prion-based disease etiology, development, and progression. Data indicates that one particular CBF is a high molecular weight component, for example, fibronectin or a member of the family of lipoproteins. See FIG. 1 (SEQ ID NO. 7, accession no. NP 002017, also see, Kornblihtt, A. R., et al. PNAS, USA 1983, 80(11):3218-22). The discovery of the interaction between the two functional ligands, i.e., CBF and NA prion-specific antibody with a prion protein opens a new direction for managing devastating diseases that involve this triumphant.

The discovery of CBF has far reaching consequences. Breeding of animals without a CBF or modified CBF will produce a breed of cows that potentially could never develop mad cow disease. On the other hand, inactivation of CBF by specially designed therapeutics should also reduce the probability of animals and humans to develop prion-based diseases.

One embodiment of the present invention describes a method for identifying the presence or absence of one or more CBFs in a sample matrix. This method comprises taking an aliquot of the sample and adding a predetermined NA antibody, such as RQ11+12 RNA, or a functional fragment thereof to the sample. (A functional fragment refers to a NA fragment that functions, e.g., binds to and interacts with a target protein as the native parent NA, but has a truncated or modified nucleotide sequence that does not affect its binding or interaction property.) To this admixture is added a predetermined protein. This protein is a known protein that forms agglomeration complexes under suitable conditions and may have at least two conformations. The first conformation of the protein is of the active protein. Preferably, the free-form of the protein is in its first conformation or active form. The second conformation is the conformation assumed by the protein in an inactive form. Preferably, in the agglomeration complex, this second, inactive conformation predominates over the first conformation. An example of such a protein is a prion protein. The admixture is then incubated under conditions suitable to allow for the agglomeration of proteins, like those conditions outlined in PCT/US02/16922.

In one aspect of the invention, the agglomeration complex comprises a protein component (“A”), a cellular binding factor (“B”), and a NA antibody (“C”), wherein the complex is represented by the formula: [AₓB_{y}C_{z}], wherein x, y, and z are integers each independently having a value from one (1) to infinity (∞). The order of constituents in the formula does not represent any particular order in the actual complex. The NA antibody (“C”) is a NA obtained from a nucleotide library comprising NA antibodies including, but not limited to, SEQ ID NOS 1-6. In one aspect, the binding forces holding the complex intact are a collection of non-covalent bonds including, but not limited to, hydrophobic, ionic, Van der Waals, hydrogen bonds, and a combination thereof.

An example of a protocol for forming an agglomeration complex is as follows: Incubate about 0.83 pmoles of human recombinant prion protein (“hrPrP”, a prion protein) with about 0.2 pmoles (or with 1.2 pmoles) of a NA antibody like RQ11+12. The RNA-protein binding is performed in 20 μl of a reaction mixture consisting of approximately 50 mM MOPS, pH 7.4; 5 mM MgCI₂; 50 mM LiCI; 1 mM DTT; 1 μg tRNA; 80-100 μg/μl BCS; 0.05% DOX, and 0.05% NP-40. The reaction mixture is incubated for about 20 minutes at around room temperature.

The prion protein employed can be hrPrP, PrPc as well as other prion proteins. The proteins will form one or more agglomerations under suitable conditions. These agglomerations can then be detected, for example, using electron microscopy. The formation of an agglomeration complex is indicative of the presence of a CBF in the reaction mixture.

The above-articulated method can be augmented by examining protease K resistance of the prion protein. In the absence of a NA antibody and/or CBF, prion proteins are soluble and readily digested by protease K. Thus, a different detection mechanism can be employed for the method presented above. Following the incubation of sample matrix with NA antibody and prion protein (PrP), proteinase K is added to the admixture. The complex formation is performed as outlined above. Resistance of PrP to protein digestion is analyzed by treating the RNA-PrP-CBF complex with protease K. For example, to the admixture, approximately 2 μg of protease K (available from commercial sources) is added to the reaction mixture and incubated for about 60 minutes at around 37° C. The reaction can be terminated using approximately 20 μL of 2× sample buffer (available from NOVEX) containing 10 mM of phosphormethylsulphonylfluoride (PMSF). Aliquots of the reaction can then be analyzed by SDS/PAGE/analysis. Samples are loaded in a 6% polyacrylamide gel with 7 M urea. Electrophoresis is performed for about 60-90 minutes at around room temperature using a 1×TBE buffer (50 mM Tris-borate, pH 8.3, 0.5% DOX, 0.5% NP-40 and 1 mM EDTA). The gel is subsequently dried and exposed to x-ray film or analyzed by phosphoimager (Bio-Rad, BIL-20). Those practitioners skilled in the art are very familiar with gel electrophoresis and alike analysis. If a Western blot is prepared, then the resistant PrP can be visualized using immuno-blot technique using, for example, monoclonal antibody (MoAb) 3F-4 that is specific for the amino acid 109-112 epitope of hamster PrP and 7A-12 MoAb specific for the amino acid 100-145 epitope of mouse PrP.

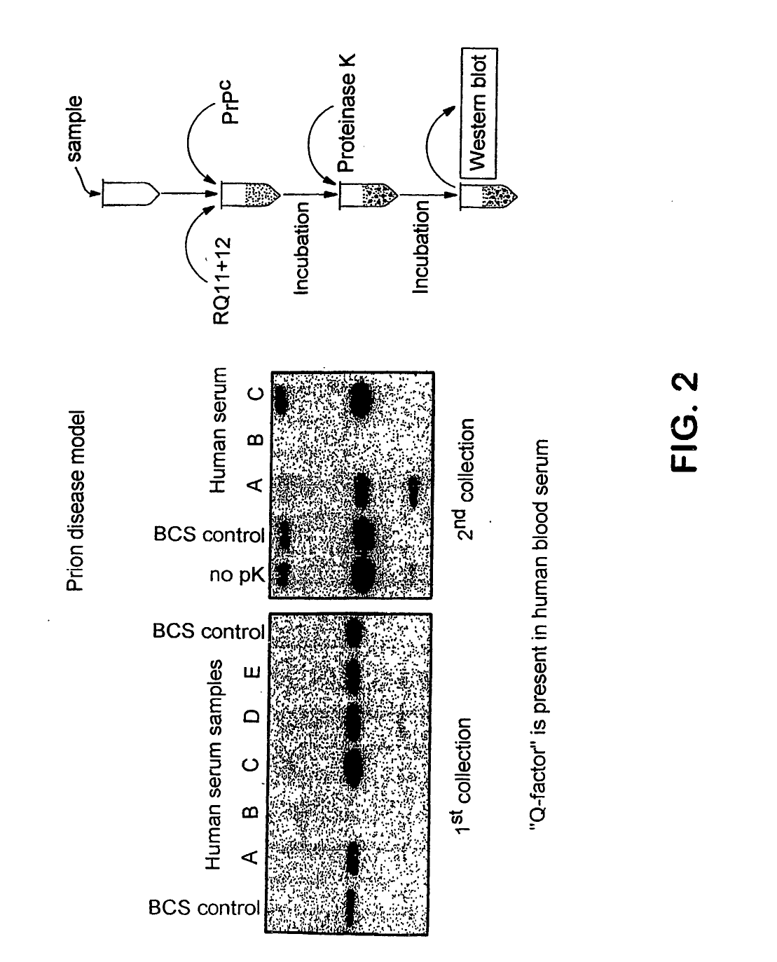

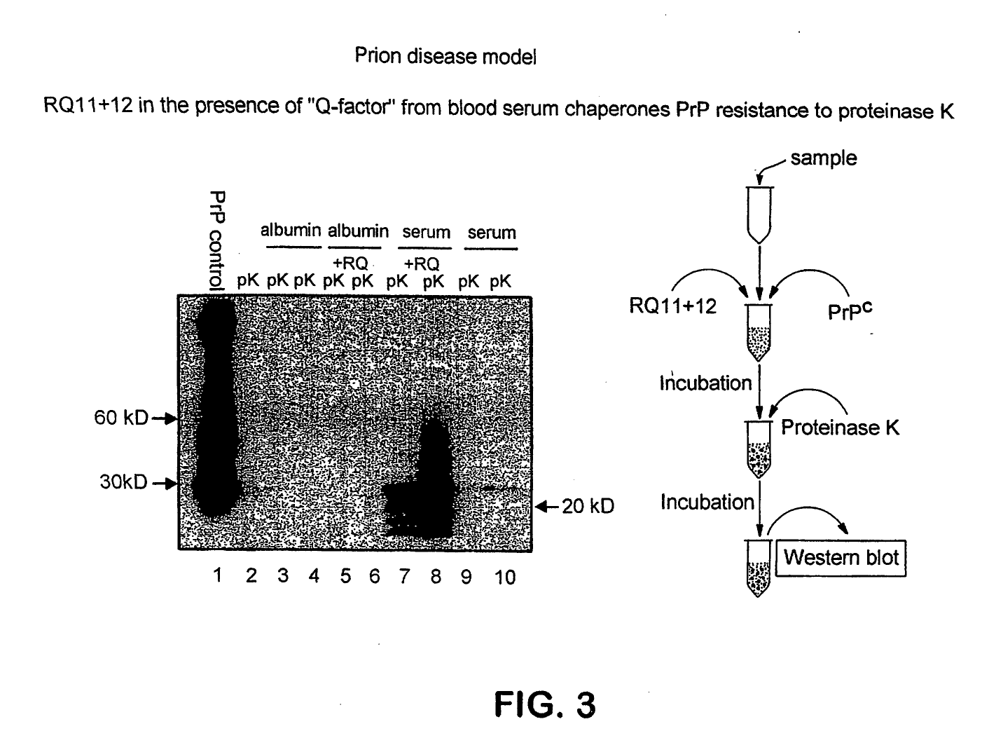

In the presence of an appropriate NA antibody and CBF, the prion protein is non-soluble and resistant to proteinase K. These prion proteins present in the mixture should agglomerate and can be detected. Therefore, protein digestion products of the prion protein will be minimal if present at all. FIGS. 2 and 3 depict typical results of such an experiment. (An example of this type of assay is described in the Example below.)

The CBF itself can be isolated from an agglomeration complex. A protein complex, of the type described herein, presumably comprises one or more NA antibodies, a protein target (such as prion protein) and one or more CBFs. The complex can be disrupted using, for example, chaotropic agents. In an experimental setting, the NA antibody and target protein will be known, thus the third major element will be the CBF. The chaotropic mixture can then be subjected to chromatography and/or gel electrophoresis employing known standards for the NA and target protein. The chromatography peak and/or electrophoresis band of unknown origin can then be isolated and further examined. In actual practice, one or more chromatographic procedures may be required to isolate and purify the CBF(s). The isolate, i.e, the putative CBF, can then be analyzed for its ability to confer, for example, proteinase K resistance upon a target protein. Thus, confirming its identity as a CBF.

The following procedure has been employed by the inventors to construct a fraction of human serum enriched with a CBF. An aliquot, e.g., 5 mL, of human plasma (no hemolysis) was mixed with 1 mL of polyethylene glycol (PEG). The mixture was allowed to stand at room temperature for approximately 15 minutes. The mixture was then centrifuged at approximately 3500×g for about 15 minutes. The supernatant was then decanted. The pellet was gently washed using approximately 2 mL of saline. The pellet was resuspended in approximately 5 mL of saline-EDTA using gentle sonication. The suspension was gently washed with approximately 2 mL saline and allowed to stand for about 2 minutes, then the supernatant was removed. The remaining pellet was resuspended with about 1 mL of saline using sonication. The suspension was dialyzed in order to remove PEG and store at 4° C.

Next, the precipitated complex was incubated in solution of 1% SDS, 50 mM DTT, 50 mM TRIS-HCl pH 6.7, 20% glycerol for approximately 10 minutes at around 95° C. Electrophoresis was then performed with a consequent extraction of the appropriate protein fraction, or the sample was applied onto a fractionation column and eluting the sample with 1% SDS, 50 mM DTT, 50 mM TRIS-HCl (pH 6.7), and 20% Glycerol.

In one embodiment of the present invention, a method for examining the efficacy of a pharmaceutical agent with respect to its ability to inhibit protein agglomeration is described. Without wishing to be bound by theory, it is believed that the sensitivity to protein digestion by a prion protein is indicative of its proclivity toward participating in protein agglomeration. It is well appreciated in the art that a protein's 3-dimensional conformation can affect the protein's susceptibility to protein digestion as well as its participation if complex formation. In the present case, a correlation has been empirically established wherein sensitivity of a prion protein to digestion by protease K is linked to prion protein agglomeration. In order for the protein to form an agglomeration complex, it must be in its insoluble conformation. In this conformation, it is insensitive to protease K digestion. In contrast, when the protein is in its soluble, free form, it is susceptible to protein digestion by, for example, protease K. Hence, if an agent can prevent the prion proteins from converting to their non-soluble conformation, then by extension of logic, this agent can prevent the formation of an agglomeration complex.

The present method comprises forming an admixture having a CBF together with a NA antibody, a prion protein, and a test pharmaceutical agent. A protease is added to this admixture, like as protease K, under conditions suitable for protein digestion. See Example C. After a suitable period of time, an aliquot is retrieved from the admixture reaction vessel and examined viz. the status of the native prion protein. If upon examination protein digestion products are detected, then this is indicative that the pharmaceutical test agent is effective in precluding the soluble prion protein from converting into its non-soluble, agglomerating conformation. Thus, the test agent is effective in preventing the formation of an agglomeration complex. However, should there be little if any digestion product upon examination, then it can be assumed that the agent is not efficacious in preventing the formation of an agglomeration complex. Moreover, an agglomeration complex should be evident upon further analysis using, for example, an electron microscope.

As an example of such analysis, a prion protein (e.g., hrPrP) is obtained and labeled using, for example, fluorescent dye Cy5 (purchased from Amersham). (Typically, the labeling protocol is provided by the vendor.) Approximately 1.6 pmoles of NA antibody like RQ11+12 RNA is incubated in a binding buffer (approximately 50 mM MOPS, pH 7,5; 1 mM EGTA; 50 mM LiCI; 5 mM MgCI₂; 1 mM DTT; 0.05% NP-40; 0.05% DOX; 5% glycerol; 1 μg BSA; 1 μg tRNA) with about 0.3 nmol of prion protein together with bovine calf serum, (about 80 μg protein) in about 10 μL of the binding buffer in the presence of a pharmaceutical test agent for about 1 hour at around ambient temperature. Then about 0.5 μg protease K is added and the mixture incubated for about 30 min. at about 37° C. The digestion reaction can be stopped by the addition of about 5 mM PMSF and about 12 μL of 2×SDS protein sample buffer. Then the reaction sample is boiled for about 10 min. at around 95° C. Samples can then be subjected to electrophoreses in PAAG (4-20%, available from NOVEX).

The present invention pertains to an assay kit. This kit can be used to assess a test agent's ability to prevent protein agglomeration. The kit comprises a NA antibody, a CBFs, and a protein. The kit can additionally comprise protease K. The NA antibody can be selected from the group consisting of SEQ ID NOS. 1-6. The CBF used in this kit can be fibronectin (e.g., SEQ ID 7) or a member of the lipoprotein family. In a particular aspect, the protein of the kit is a prion protein. Preferably, this kit is used in conjunction with the methods described herein. (Also, see Example C.)

The features and other details of the invention will now be more particularly described and pointed out in the examples. It will be understood that the particular embodiments of the invention are shown by way of illustration and not as limitations of the invention. The principle features of this invention can be employed in various embodiments without departing from the spirit and scope of the invention.

### EXAMPLES

A. Protease K Protection

FIG. 2 is a gel demonstrating the resistance of a prion protein to Protease K in the presence of CBF and a NA antibody. Seven pmoles of hrPrP was incubated with 123 fmoles of the RQ 11+12 RNA (“RQ RNA”) in 10 μL binding buffer for 17 hours at room temperature. Next, the protease K (50 ng/μL) was added and the sample was incubated for 30 minutes at 37° C. The proteolysis was terminated by the addition of PMSF (5 mM) and 10 μL of 2×SDS sample buffer. The sample was then incubated for 7 minutes at 95° C. and analyzed by PAGE and immunoblotted using 3F-4 anti PrP antibody (available from Q-RNA).

Western immunoblotting data using anti-PrP monoclonal antibody (3F-4) illustrates that hrPrP can be protected from protease K digestion if the CBF is present in the RQ RNA-PrP admixture during complex formation. The CBF was in fact present in the bovine calf serum (“BCS”).

B. Presence of CBF in Human Serum

FIG. 3 depicts two gels demonstrating the presence of a CBF in human serum. RQ RNA was incubated in 10 μL of a binding buffer (50 mM MOPS, pH 7,5; 1 mM EGTA; 50 mM LiCI; 5 mM MgCI₂; 1 mM DTT; 0.05% NP-40; 0.05% DOX; 5% glycerol; 1 μg BSA; and 1 μg tRNA) with 250 ng of hrPrP in the presence of human or bovine calf serum (80 μg protein) for 1 hour at room temperature. Then 0.5 μg protease K was added and incubated for 30 minutes at 37° C. The digestion was arrested by the addition of PMSF (5 mM) and 12 μL of 2×SDS protein sample buffer. Samples were then heated for 10 minutes at 95° C. Samples were then run in PAAG (4-20%, NOVEX) and immunoblotted using 3F-4 monoclonal Antibody to hrPrP. Western immunoblotting with anti-PrP monoclonal antibody (3F-4) data demonstrated that different preparations of human serum have a unique ability to generate the resistance of hrPrP to protease K digestion indicating the presence of CBF in these samples.

C. PrP Protection Assay

Human recombinant prion (hrPrP), refolded from Guanidine-HCl-SDS solution, was used for labeling with fluorescent dye Cy5 (purchased from Amersham). (Labeling protocol is provided by the vendor.) Approximately 1.6 pmoles of RQ11+12 RNA was incubated in a binding buffer (50 mM MOPS, pH 7,5; 1 mM EGTA; 50 mM LiCI; 5 mM MgCI₂; 1 mM DTT; 0.05% NP-40; 0.05% DOX; 5% glycerol; 1 μg BSA; 1 μg tRNA) with 0.3 nmol of hrPrP together with bovine calf serum, (80 μg protein) in 10 μL of the binding buffer in the presence of chlorpromazine for 1 hour at ambient temperature. Then 0.5 μg protease K were added and the mixture was incubated for 30 min. at 37° C. The digestion reaction was arrested by the addition of 5 mM PMSF and 12 μl of 2×SDS protein sample buffer. Then the reaction sample was boiled for 10 min. at 95° C. Samples were then subjected electrophoreses in PAAG (4-20%, NOVEX).

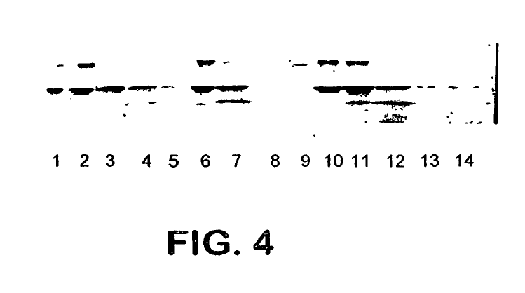

FIG. 4 depicts the efficacy of chlorpromazine in blocking the PrP protection by serum or lipoproteins in a dose-dependent manner. Specifically, lanes 1, 6, and 10 represent reactions in which there was no protease treatment; lanes 2, 3, 4, 5, 7, 8, 9, 11, 12, 13, and 14, represent protease K treated samples; lanes 3, 7, 12 represent samples having the addition of 20 mM of chlorpromazine; lanes 4, 8,13 represent samples having the addition of 40 mM chlorpromazine; lanes 5, 9, and 14 represent reactions in which 80 mM chlorpromazine was added. Finally, lanes: 1-5, represent the HS fraction of blood serum; 6-9, represent the LP1 fraction of blood serum; 10-14, represent the LP2 fraction of blood serum. It can be observed that in the presence of chlorpromazine, prion protein's sensitivity to protease K digestion is preserved.

All of the reagents mentioned herein can be obtained from commercial sources or through Q-RNA of New York, N.Y.

While this invention has been particularly shown and described with reference to embodiments thereof, it will be appreciated by those skilled in the art that various changes in form and detail may be made therein without departing from the spirit and scope of the invention as defined by the appended claims hereto.

What is claimed is:

1. A method of identifying the presence or absence of an agglomeration complex from a sample matrix obtained from an individual comprising the following steps:
(a) forming an admixture of said sample matrix with one or more nucleic acids, wherein said one or more nucleic acids are obtained from a nucleotide antibody library;
(b) incubating said admixture of step (a) under conditions suitable to form at least one agglomeration complex, wherein said agglomeration complex is represented by the following formula:
[AₓB_{y}C_{z}]
wherein A represents a protein and x is an integer having a value from one to infinity, B is a cellular binding cofactor, wherein said binding cofactor participates in the formation of the complex, and y is an integer having a value from 1 to infinity, and C is a nucleic acid selected from said library of nucleic acid antibodies and z is an integer having a value from 1 to infinity, wherein at least one of the groups consisting of A and B are present in said sample matrix, and the complex represented in brackets does not imply any order in A, B, or C, and A, B and C form an agglomeration complex through non-covalent bonds;
(c) detecting said agglomeration complex.

2. The method of claim 1, wherein said agglomeration complex exhibits decreased solubility in which said sample matrix is obtained from an individual affected with a prion-based disease.

3. The method of claim 1, wherein said agglomeration complex exhibits increased stability in the presence of proteolytic enzymes.

4. The method of claim 3, wherein said proteolytic enzyme is protease K.

5. The method of claim 1, wherein said agglomeration complex is isolated from said sample matrix to form an isolation product.

6. The method of claim 5, wherein said agglomeration complex is separated into components A, B and C.

7. The method of claim 6, wherein said components A, B, and C are identified and compared to components derived from a second sample matrix obtained from a second individual who is unaffected by a prion-based disease.

8. The method of claim 1, wherein said nucleotide antibody library is derived from naturally occurring NA.

9. The method of claim 1, wherein said nucleotide antibody library is derived from non-naturally occurring NA.

10. The method of claim 9, wherein said nucleotide antibody library comprises RQ11+12, MDV, MNV, MNV-AP1, MNVUP, MNVLO RNA, and combinations thereof.

11. The method of claim 1, wherein said nucleotide antibody library is derived from an individual exhibiting symptoms of a prion-based disease.

12. The method of claim 1, wherein said protein has at least two functional conformations, a first active conformation, and a second inactive conformation.

13. The method of claim 1, wherein said cellular binding factor is selected from the family of lipoproteins.

14. The method of claim 1, wherein said cellular binding factor is fibronectin.

15. The method of claim 1, wherein said protein is human recombinant prion protein.

16. A composition associated with an agglomeration complex, wherein said agglomeration complex is represented by the following formula:
[AₓB_{y}C_{z}]
wherein A represents a protein and x is an integer having a value from one to infinity, B is a cellular binding cofactor, wherein said binding cofactor participates in the formation of the complex through non-covalent interactions with macromolecules, and y is an integer having a value from 1 to infinity, and C is a nucleic acid selected from a library of nucleic acid antibodies and z is an integer having a value from 1 to infinity, wherein at least one of the groups consisting of A and B are present in said sample matrix, and wherein the complex represented in brackets does not imply any order in A, B, or C, and A, B and C form a complex through non-covalent bonds and non hybridization affinity.

17. The composition of claim 16, wherein at least one of the group consisting of A and B are present in a sample matrix of an individual exhibiting symptoms of the disease from which the agglomeration complex is associated.

18. The composition of claim 16, wherein said agglomeration complex exhibits decreased solubility in which said sample matrix is obtained from an individual affected with a prion-based disease.

19. The composition of claim 16, wherein A is a prion-protein.

20. The composition of claim 16, wherein said nucleotide antibody library comprises RQ11+12, MDV, MNV, MNV-AP1, MNVUP, MNVLO RNA and combinations thereof.

21. A method for examining the efficacy of a pharmaceutical agent to protect prion protein against protease K digestion, comprising the following steps:
(a) forming an admixture under having
(i) one or more cellular binding factors;
(ii) one or more nucleic acid antibodies, wherein said one or more nucleic acids antibodies are obtained from a nucleotide antibody library;
(iii) one or more prion proteins; and
(iv) said pharmaceutical agent;
(b) adding to said admixture of step (a) protease K under conditions suitable
for protease digestion of said prion protein;
(c) detecting the presence of said prion protein of step (a), whereby the presence of said prion protein is indicative of a pharmaceutical agent that is effective in protecting said prion protein against protease digestion by protease K.

22. The method of claim 21, wherein said nucleotide antibody library is derived from naturally occurring NA.

23. The method of claim 21, wherein said nucleotide antibody library is derived from non-naturally occurring NA.

24. The method of claim 21, wherein said nucleotide antibody library comprises RQ11+12, MDV, MNV, MNV-AP1, MNVUP, MNVLO RNA, and combinations thereof.

25. The method of claim 21, wherein said cellular binding factor is selected from the family of lipoproteins.

26. The method of claim 21, wherein said cellular binding factor is fibronectin.

27. The method of claim 21, wherein said prion protein is human recombinant prion protein.

28. The method of claim 21, wherein said pharmaceutical agent is chlorpromazine.

29. A kit for determining whether a test agent can inhibit protein agglomeration, comprising:
(a) a NA antibody;
(b) a CBF; and
(c) a protein.

30. The kit of claim 29, wherein said NA antibody is a nucleotide sequence selected from group consisting of SEQ ID NOS 1-6.

31. The kit of claim 29, wherein said CBF is fibronectin.

32. The kit of claim 29, wherein said protein is a prion protein.

33. The kit of claim 29 further comprising protease K.
